(19) United States
(12) Patent Application Publication
(10) Pub. No.: US 2026/0306314 A1
(43) Pub. Date: Oct. 8, 2026

(54) PACKAGE COMPRISING A BAG AND A STACK OF ABSORBENT ARTICLES AND MANUFACTURING METHOD THEREOF

(71) Applicant: Drylock Technologies NV, Zele (BE)

(72) Inventors: Pieter Hommez, Zele (BE); Rik Daelemans, Zele (BE); Werner Van Ingelgem, Zele (BE); Steven Smet, Zele (BE); Bart Van Malderen, Zele (BE)

(21) Appl. No.: 19/483,206

(22) PCT Filed: May 23, 2024
(86) PCT No.: PCT/EP2024/064238

§ 371 Date: Nov. 10, 2025

(30) Foreign Application Priority Data
May 23, 2023 (NL) 2034899

(51) Int. Cl. A61F 13/551; A61F 13/15; B65B 5/06; B65B 7/02

(52) U.S. Cl. CPC A61F 13/5511; A61F 13/15747; A61F 13/55115; B65B 5/067; B65B 7/02

(57) ABSTRACT

A package comprising a bag and a stack of absorbent articles having a substantially rectangular shape, wherein the stack of absorbent articles is arranged in the bag, wherein the bag is made of a paper material, wherein the bag has a closed bottom, a closed top, at least one outer attached portion, and a peripheral wall between said bottom and said top, wherein the bag is provided at least on an inner surface of the bag near the top, with a first sealable coating, wherein the closed top is realized by a sealed portion of said first sealable coating, wherein the bag is provided at least on an outer surface of the bag near the top with a second sealable coating wherein the at least one outer attached portion is realized by a sealed portion of the second sealable coating.

### TECHNICAL FIELD

The present invention pertains to the technical field of absorbent articles, and more in particular to packages comprising a bag and a stack of absorbent articles. The present invention also pertains to methods for manufacturing such packages for a stack of absorbent articles.

### BACKGROUND

In recent years there has been a development towards the manufacturing of eco-friendly absorbent articles wherein less chemicals and more sustainable materials are used as compared to traditional disposable absorbent articles.

Typically, the packaging of absorbent articles is made of plastic material. Plastic packaging material is very flexible to be manipulated into different shapes and sizes of packaging, can be easily sealed and offers a lot of possibilities in view of coloring and/or branding of the absorbent article packaging. Although more eco-friendly, non-plastic packaging materials exist, these materials unfortunately do not provide the same benefits in view of flexibility and/or usability in terms of packaging as compared to plastic packaging materials.

### SUMMARY

The object of embodiments of the invention is to provide a package of absorbent articles which is both eco-friendly and user friendly. More in particular it is an object of embodiment of the invention to provide a package which offers protection against environmental influences to the absorbent articles, which can easily be handled by a user, which is visually appealing and/or which can easily be opened by a caregiver when needed.

According to a first aspect there is provided a package comprising a bag and a stack of absorbent articles having a substantially rectangular shape, wherein the stack of absorbent articles is arranged in the bag. The bag is made of a paper material. The bag has a closed bottom, a closed top, at least one outer attached portion and a peripheral wall between said bottom and said top, and the bag is provided at least on an inner surface of the bag near the top, with a first sealable coating. The closed top is realized by a sealed portion of the first sealable coating. The bag is provided at least on an outer surface of the bag near the top with a second sealable coating. The at least one outer attached portion is realized by a sealed portion of the second sealable coating.

Embodiments of the invention are based on the inventive insight that by providing a first sealable coating, e.g. a thermoplastic coating, on the inner surface of the bag near the top, the paper material bag can be efficiently sealed.

It is noted that the first sealable coating can be provided only locally near the top, or alternatively over a larger portion of the inner surface or over substantially the entire inner surface of the bag.

The first sealable coating can be applied according to a continuous or discontinuous coating pattern.

In addition, by providing a second sealable coating, e.g. a thermoplastic coating, on the outer surface of the bag near the top, outer parts of the paper material bag can be efficiently attached to each other. In this manner, the paper material bag can be further designed to be visually appealing and/or more user friendly.

It is noted that the second sealable coating can be provided only locally near the top, or alternatively over a larger portion of the outer surface, especially on at least part of an area of a side wall. The second sealable coating can be applied according to a continuous or discontinuous coating pattern.

It is noted that the bag, alternatively or in addition to comprising paper material, can comprise other non-plastic materials such as non-woven materials. Preferably the bag is made of a non-plastic material. In the context of this application a paper material is to be understood as a material produced by pressing together moist fibres of cellulose pulp, optionally in combination with other materials, and drying the fibres into preferably flexible sheets. Preferably the paper material is a single-layered material. Alternatively, the paper material is a laminated paper material. Single-layered material is preferred over laminated paper material as it does not require additional glue or adhesive to laminate at least two layers of the laminated paper material.

Preferably the first sealable coating is water based. In less preferred embodiments the coating may be acrylic or PE based. Preferably the coating is recyclable, e.g. in accordance with UNI 11743:2019. Preferably the coating is mineral-oil free, and more preferably fulfils the standard FDA 175.105-176.180. Preferably, the coating has a seal temperature initiation between 70 and 110° C.

Preferably the second sealable coating is water based. In less preferred embodiments the coating may be acrylic or PE based. Preferably the coating is recyclable, e.g. in accordance with UNI 11743:2019. Preferably the coating is mineral-oil free, and more preferably fulfils the standard FDA 175.105-176.180. Preferably, the coating has a seal temperature initiation between 70 and 110° C.

According to an exemplary embodiment, the peripheral wall comprises a front wall, a rear wall opposite said front wall, a first side wall, and a second side wall opposite said first side wall, wherein the second sealable coating is provided on the first side wall or the second side wall to realize the at least one outer attached portion on the first side wall or the second side wall, respectively.

Preferably, the first side wall or the second side wall is folded inwardly at the location of the second sealable coating such that two first folded portions are formed where the top joins the first side wall or such that two second folded portions are formed where the top joins the second side wall. The at least one outer attached portion is realized by a sealed portion of the second sealable coating attaching the two first folded portions together or attaching the two second folded portions together, respectively.

Preferably, at the top, the other one of the first and the second side wall, i.e. the side wall not comprising the second sealable coating, is folded inwardly such that two first wing portions are formed where the top joins the first side wall or such that two second wing portions are formed where the top joins the second side wall.

Alternatively, or in addition the second sealable coating can be dimensioned as to only realize one or more partial outer attached portion(s), i.e. wherein only corresponding parts of the first two wing portions are adhered to each other, and/or wherein only corresponding parts of the second two wing portions are adhered to each other.

As will be further described in combination with the figures, the paper bag typically enwraps the stacked absorbent articles quite closely or narrowly. This, in combination with the higher stiffness of paper material as compared to plastic packaging material, results in a rather rigid package which is not easily pressed in, e.g. by a finger or a hand, and thus cannot be easily gripped. However, the formed wing portions provide gripping means for a user and allow the package of absorbent articles to be easily gripped. Since preferably two wing portions are provided near each side wall, the resulting package can be easily gripped or handled from many different directions and according to many different orientations. It is further noted that the provision of the wing portions also results in a dented corner area below the wing portions at each side wall, where the package can be easily gripped and the bag can be opened by pulling “open” the wing portions. In addition the resulting package can be stacked and/or displayed in a supermarket on any one of the faces of the package, e.g. on the bottom, on the top, or on any one of the sides of the peripheral wall, i.e. front, rear, first side, second side.

In further exemplary embodiments the length of the sealed first wings and the length of the sealed second wings is between 1 cm and 8 cm, preferably between 2 cm and 7 cm, more preferably between 3 cm and 6 cm. It has been found that wings having these dimensions are especially convenient to be gripped by shoppers and/or caregivers.

In further developed exemplary embodiments the length of the sealed portion between the first wings and the second wings is between 8 cm and 40 cm, preferably between 10 cm and 30 cm, more preferably between 12 cm and 20 cm.

According to an exemplary embodiment, the peripheral wall comprises a front wall, a rear wall opposite said front wall, a first side wall, and a second side wall opposite said first side wall. The at least one outer attached portion comprises a first outer attached portion and a second outer attached portion. The second sealable coating is provided on the first side wall and the second side wall to realize the first outer attached portion on the first side wall and the second attached portion on the second side wall.

Preferably, the first side wall or/and the second side wall is folded inwardly at the location of the second sealable coating such that two first folded portions are formed where the top joins the first side wall or/and such that two second folded portions are formed where the top joins the second side wall. The at least one outer attached portion is realized by a sealed portion of the second sealable coating attaching the two first folded portions together or/and attaching the two second folded portions together, respectively.

Alternatively, or in addition the second sealable coating can be dimensioned as to only realize one or more partial outer attached portion(s), i.e. wherein only corresponding parts of the two first folded portions are adhered to each other, and/or wherein only corresponding parts of the two second folded portions are adhered to each other.

Preferably the paper material has a basis weight between 40 and 100 g/m2, more preferably between 50 and 90g/m2, even more preferably between 60 and 80g/m2, measured according to the ISO 536 standard. In this manner the paper material bag provides a sufficient amount of protection against environmental influences on the stack of absorbent articles. In this manner, the absorbent articles are kept safe from any possible pollution or contamination before the package is actually opened. Although paper material is less flexible as compared to the commonly used plastic packaging material, it has been found that this particular range of basis weight allows that the paper material is manipulated in such a way as to fold and/or shape it into a bag, without the risk of the paper material being ripped or torn.

According to a further embodiment, the first sealable coating extends at least over a closed peripheral inner portion of the inner surface of the bag. In this manner a good and tight seal of the package is ensured. In addition this allows that each wing portion or folded portion that is formed can also be adequately sealed at the inner side thereof.

Preferably, the second sealable coating is present on the outer surface of the bag at least at the level of the first sealable coating of the wing portion or folded portion, wherein the first sealable coating of the wing portion or folded portion is present on the inner surface of the bag. In other words, at such location the paper material is located between the first sealable coating at the inside and the second sealable coating at the outside. The first sealable coating is configured to provide a sealing between two inner surfaces of the bag, whereas the second sealable coating is configured to provide sealing between two outer surfaces of the bag.

Preferably, the stack of absorbent articles is arranged such that each absorbent article extends in an upright direction between the bottom and the top. In this manner, when the package of absorbent articles is opened near the top, the absorbent articles can easily be taken out of the package.

According to typical embodiments, the absorbent articles are any one of the following: baby diapers, pants, adult incontinence garments.

Preferably, the absorbent article is a folded absorbent article, wherein a crotch portion of the absorbent article is located at the top of the bag, and wherein a rear and front end of the absorbent article are located at the bottom of the bag. In this manner, when the bag is opened near the sealed top side a caregiver can easily take one individual absorbent article out of the bag, while keeping the other absorbent articles intact and in the bag. Although less preferred, embodiments exist wherein the absorbent articles are folded and/or arranged in the bag in a different manner.

Preferably, the folded absorbent articles have a width between 7 cm and 20 cm, and a height between 15 cm and 40 cm.

According to an embodiment, the bottom has a width between 9 cm and 20 cm, preferably between 10 cm and 15 cm, and a length between 15 cm and 50 cm, preferably between 20 cm and 40 cm.

According to exemplary embodiment, the coating is made of any one of the following materials or a combination thereof: a polymer material such as a polyethylene material, e.g. as a low density polyethylene (LDPE), a bio coating, a printed lacquer, 1,4 succinic, fumaric and malic acids, 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinito, tricarboxylic acid. In this manner a coating is provided which can be efficiently sealed by applying heat and/or pressure thereto. Preferably the applied first sealable coating is biodegradable.

Preferably, the coating has a basis weight between 3 g/m² and 25 g/m², more preferably between 5 g/m² and 25 g/m² , even more preferably between 7 g/m² and 20 g/m², even more preferably between 8 g/m² and 16 g/m².

Preferred coating materials include, but are not limited to, LDPE, preferably having a basis weight between 3 g/m² and 6 g/m², biocoating, preferably having a basis weight between 13 g/m² and 17 g/m².

According to a preferred embodiment the paper material has a basis weight measured according to the ISO 536 standard between 65 g/m² and 85 g/m². It has been found that this particular range of basis weight allows that the paper material is manipulated in such a way as to fold and/or shape it into a bag, while reducing the risk that the paper material is ripped or torn during manufacturing of the package.

Preferably the paper material has a basis weight of about 70 g/m², most preferably the paper material has a basis weight of about 80 g/m².

It is clear to the skilled person that paper material having the required basis weight can be combined with one or more of the described coating materials, and that many combinations are possible.

Preferably the length of the attached first folded portions and the length of the attached second folded portions is between 1 cm and 8 cm, preferably between 2 cm and 7 cm, more preferably between 3 cm and 6 cm.

Preferably the length of the sealed portion between the attached first folded portions and the attached second folded portions is between 8 cm and 40 cm, preferably between 10 cm and 30 cm, more preferably between 12 cm and 20 cm.

According to a further embodiment the bottom is formed by folding and gluing a portion of the paper material, such that is has a substantially rectangular shape. Such substantially rectangular shaped bottom is preferred to support the stack of absorbent articles in the bag in terms of efficient use of space and/or material.

Preferably, the bottom is provided with a handle made of paper material. In this manner, the package can be even more easily gripped.

Preferably, the package further comprises a paper sheet with two cuts wherein the paper sheet is glued to the bottom of the bag in an area around the two cuts, such that an area of the paper sheet between the two cuts functions as the handle.

According to a possible embodiment the paper material is an unbleached paper material. This contributes to the eco-friendly character of the packaging, since fewer chemicals are used in manufacturing the packaging as compared to bleached paper. Preferably, the paper material is a full brown paper material, such as a calendared brown paper material, having a basis weight according to the ISO 536 standard between 65 g/m² and 85 g/m². More preferably the paper material has a basis weight of about 70 g/m², most preferably the paper material has a basis weight of about 80 g/m².

According to a preferred embodiment the paper material has any one or more of following properties, in test conditions as specified in ISO 554-1976 (23+/−1 ° C./50+/−2%):
a tensile strength measured according to the ISO 1924-3 standard in a machine direction between 4 and 10 kN/m, preferably between 5 and 7 kN/m, and in a cross direction between 2 and 6 kN/m, preferably between 4 and 5 kN/m;
a tear index measured according to the ISO 1974 standard in a machine direction between 8 and 16mN.m2/g, and in a cross direction between 9 and 17 mN.m2/g;
a tear measured according to the ISO 1974 standard in a machine direction between 400 and 1300 mN, and in a cross direction between 450 and 1450 mN.

It has been found that any of these properties enable the paper material to be manipulated during a manufacturing process of a package filled with absorbent articles, while reducing the risk that the paper material is prematurely ripped or torn.

According to another aspect of the present invention, there is provided a method for manufacturing a package for a stack of absorbent articles, comprising the steps of:
arranging the stack of absorbent articles in a bag made of a paper material having a basis weight measured according to the ISO 536 standard between 60 and 100 g/m2, wherein the bag has a closed bottom, an open top, and a peripheral wall between the bottom and the top, and wherein the bag is provided at least on an inner surface of the bag near the top, with a first sealable coating; and
sealing a portion of said first sealable coating to obtain a closed top end; and
sealing a portion of said second sealable coating to obtain at least one outer attached portion.

It will be understood by the skilled person that the features and advantages disclosed hereinabove with respect to various embodiments of the package may also apply, mutatis mutandis, to various embodiments of the manufacturing method.

According to an embodiment, the peripheral wall comprises a front wall, a rear wall opposite to the front wall, a first side wall, and a second side wall opposite to the first side wall. Preferably, before the step(s) of sealing at the first and/or second sealable coating, the first or/and the second side wall are folded inwardly at the top, such that two first folded portions are formed where the top joins the first side wall or/and such that two second folded portions are formed where the top joins the second side wall, and wherein the at least one outer attached portion is realized by a sealed portion of the second sealable coating attaching the two first folded portions together or/and attaching the two second folded portions together, respectively.

According to a preferred embodiment, before the step(s) of sealing at the first and/or second sealable coating, the open top end of the bag is provided with a manipulation part containing a plurality of holes, and, prior to the step of arranging of the stack of absorbent articles in the bag, the bag is arranged on a stack of bags, with a plurality of pins extending through the plurality of holes. Preferably, the step of arranging comprises opening the bag whilst the bag is held by the plurality of pins, and pushing the stack of absorbent articles in the bag. A hole may comprise e.g. a round hole with a diameter e.g. between 10 and 30 mm. The skilled person understands that other shapes (e.g. oval, polygonal, such as rectangular or square, etc) and sizes are also possible.

Preferably, the manipulation part containing the plurality of holes is a part made of a paper material, e.g. a paper material having a basis weight between 60 and 100 g/m2 measured according to the ISO 536 standard, or of a polymer material, e.g. polyethylene material such as a low density polyethylene material.

Preferably, the manipulation part is a separate part glued to the bag or an integral part of the bag.

Preferably, the manipulation part is substantially rectangular and has a length between 20 cm and 35 cm and a width between 4 cm and 15 cm.

According to an exemplary embodiment, the method further comprises, after or during the step of sealing, the step of cutting of an end part of the top of the bag.

Preferably, the end part comprises the manipulation part containing the plurality of holes. According to an embodiment, the stack of absorbent articles is arranged in the bag such that each absorbent article extends substantially perpendicular to the closed bottom of the bag.

Preferably, the closed bottom is formed by folding and gluing a portion of the paper material, such that is has a substantially rectangular shape.

According to typical embodiments, the absorbent articles are any one of the following: baby diapers, pants, adult incontinence garments.

According to a preferred embodiment, the paper material has a basis weight measured according to the ISO 536 standard between 65 and 85 g/m2.

According to an embodiment the coating has a basis weight between 5 g/m2 and 25 g/m2 , preferably between 7 g/m2 and 20 g/m2 , more preferably between 8 g/m2 and 16 g/m2 .

According to a further embodiment, the bottom or the top is provided with a handle.

According to a further developed embodiment, the method further comprises providing a paper sheet with two cuts, and gluing said paper sheet to the bottom of the bag in an area around the two cuts, such that an area of the paper sheet between the two cuts functions as the handle.

According to a preferred embodiment the absorbent articles are folded absorbent articles having a width between 7 cm and 20 cm, and a height between 15 cm and 40 cm.

Preferably, the plurality of pins comprises a plurality of retarding elements, wherein during opening of the bag the plurality of holes of the manipulation part are guided over the plurality of retarding elements. When the manipulation part is pulled up and the plurality of holes are guiding along the plurality of retarding elements, the manipulation part and the part of the bag to which the manipulation part is attached is retarded or slowed down during the upward movement thereof.

This causes the bag to be opened in a controlled manner and reduces the risk of premature and/or uncontrolled tearing or ripping of the bag during opening of the bag.

Preferably, each retarding element of the plurality of retarding elements comprises a first portion having a first cross-section and a second portion having a second cross-section, wherein the first cross-section is smaller than the second cross-section. In this manner the retarding element comprises different portions with corresponding different guiding properties for the plurality of holes. Preferably the plurality of holes is dimensioned in such a way that the manipulation part will be more easily guided along the first portion as compared to the second portion.

Preferably, during opening of the bag, each hole of the plurality of holes is guided over the first portion of the respective retarding element before being guided over the second portion of the respective retarding element. In this manner, the manipulation part and the corresponding part of the bag is efficiently retarded.

Preferably, a cross-section of the retarding element gradually increases from the first cross-section to the second cross-section.

In a preferred embodiment, the first portion is a cylindrical portion having a first diameter, and the second portion is a conical portion having a diameter which increases from the first diameter to a second larger diameter, wherein the manipulation part is first guided along the first portion and next along the second portion. More preferably, the plurality of holes comprises a plurality of round holes having a diameter between the first and the second diameter.

Any of the features described above in connection with the package of absorbent articles are also applicable for embodiments of the method.

### BRIEF DESCRIPTION OF FIGURES

The accompanying drawings are used to illustrate presently preferred non-limiting exemplary embodiments of devices of the present invention. The above and other advantages of the features and objects of the invention will become more apparent and the invention will be better understood from the following detailed description when read in conjunction with the accompanying drawings, in which:

FIG. 1 is a perspective view of an open bag and a stack of absorbent articles before a package according to an embodiment is formed;

FIG. 2 is a perspective view of a package comprising a bag and a stack of absorbent articles according to an embodiment;

FIG. 3 illustrates a package for a stack of absorbent articles according to an embodiment in the process of being manufactured;

FIGS. 4A-4D schematically illustrate a method for manufacturing a package for a stack of absorbent articles according to an embodiment wherein the second sealable coating is not illustrated;

FIG. 5 illustrates a further embodiment of a package comprising a bag and a stack of absorbent articles; and FIG. 6 illustrates another embodiment of a package comprising a bag and a stack of absorbent articles;

FIGS. 7A-7D schematically illustrate steps of a method for manufacturing a package for a stack of absorbent articles according to an embodiment wherein the second sealable coating is not illustrated; and

FIGS. 8A-8C illustrate a retarding element according to different embodiments.

### DESCRIPTION OF EMBODIMENTS

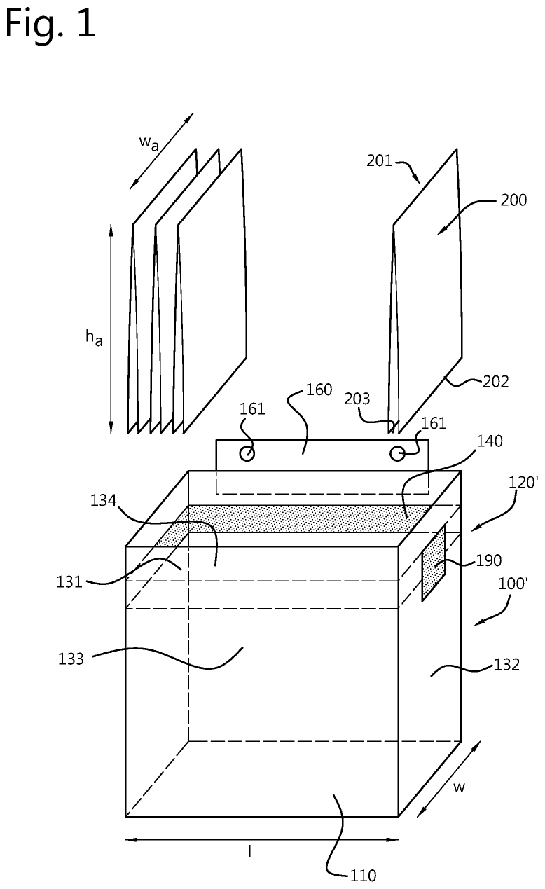

FIG. 1 illustrates an open bag 100′ and a stack of absorbent articles 200 to be inserted in the open bag 100′. The bag 100′ is made of a non-plastic material and preferably has a basis weight between 60 and 100 g/m². In a preferred embodiment the bag 100′ is made from paper material, but alternative embodiments exist wherein the bag is made from other non-plastic material, such as non-woven material. The bag 100′ has a bottom 110 and a peripheral wall 130 extending upwardly from the bottom 110 leading up to an open top 120′. The peripheral wall 130 comprises a front wall 133, a rear wall 134 positioned opposite to the front wall 133, a first side wall 131, and a second side wall 132 positioned opposite to the first side wall 131. The illustrated bottom has the shape of a rectangle wherein a length dimension l is larger than a width dimension w. Preferably the bottom has a length l between 15 cm and 50 cm, preferably between 20 cm and 40 cm, and a width w between 9 cm and 20 cm, more preferably between 10 cm and 15 cm. In alternative embodiments the bottom may have the shape of a square or another geometric figure. However, the illustrated substantially rectangular shaped bottom is preferred in terms of filling efficiency and further shaping of the bag 100′. For example the bottom 110 can be formed by folding and gluing a portion of the non-plastic material, such that is has a substantially rectangular shape. Optionally, the bottom is provided with a handle made of paper material. For example, the package may further comprising a paper sheet with two cuts, wherein the paper sheet is attached, e.g. glued to the bottom of the bag in an area around the two cuts, such that an area of the paper sheet between the two cuts functions as the handle.

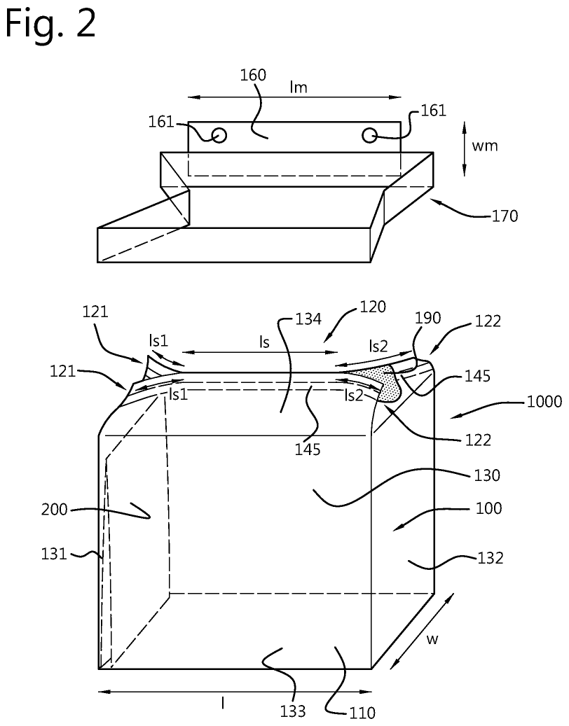

At least near the open top 120′ the bag is provided with a first sealable coating 140 which is configured to be sealed at least along a portion thereof to form a closed top 120 of the bag, as is further illustrated in FIG. 2. Preferably the first sealable coating 140 is a heat sealable coating and comprises any one of the following materials or a combination thereof: a polymer material such as a polyethylene material, e.g. as a low density polyethylene (LDPE), a bio coating, a printed lacquer, 1,4 succinic, fumaric and malic acids, 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinito, tricarboxylic acid. Preferably the first sealable coating 140 is provided on an inner surface of the peripheral wall 130. However, alternatively or in addition the first sealable coating can be provided on an outer surface of the bag 100′. In the illustrated embodiment of FIG. 1 the first sealable coating 140 is provided in the shape of a substantially continuous strip which extends over the front wall 133, the first side wall 131, the rear wall 134 and the second side wall 132 to form a closed loop on the inner surface of the bag 100′. In other words, the first sealable coating 140 extends over a closed peripheral inner portion of the inner surface of the bag 100′. However, alternatively or in addition the first sealable coating 140 may be provided on a larger portion of the inner or outer surface of any one of the front wall 133, the first side wall 131, the rear wall 134, the second side wall 132, and a combination thereof. In addition or alternatively to the substantially continuous shape of the first sealable coating 140, the first sealable coating 140 can be applied according to a discontinuous coating pattern. Such discontinuous coating pattern may comprise multiple lines, strips, dots or other geometric shapes which may at least partially overlap when viewed in a height direction of the bag 100′ such that the open top 120′of the bag can be adequately sealed along at least a portion of the first sealable coating 140 to form a closed top 120. Preferably, the first sealable coating 140 has a basis weight between 5 g/m² and 25 g/m², more preferably between 7 g/m² and 20 g/m², and even more preferably between 8 g/m² and 16 g/m².

At least on an outer surface near the open top 120′ the bag is provided with a second sealable coating 190 which is configured to be sealed at least along a portion 195 to realize at least one outer attached portion 180 wherein two outer areas of the bag are adhered to each other by means of the second sealable coating 190, as will be further described in view of FIGS. 2 and 6.

Preferably the second sealable coating 190 is a heat sealable coating and comprises any one of the following materials or a combination thereof: a polymer material such as a polyethylene material, e.g. as a low density polyethylene (LDPE), a bio coating, a printed lacquer, 1,4 succinic, fumaric and malic acids, 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinito, tricarboxylic acid. Preferably the second sealable coating 190 is provided on an outer surface of the peripheral wall 130. In the illustrated embodiment of FIG. 1 the second sealable coating 140 is provided in the shape of a substantially continuous rectangular strip which is positioned on at least a central part of the second side wall 132 leading up to the open top 120′. However, alternatively or in addition the second sealable coating 190 may be provided on a larger portion of the outer surface of any one of the front wall 133, the first side wall 131, the rear wall 134, the second side wall 132, and a combination thereof. In addition or alternatively to the substantially continuous shape of the second sealable coating 190, the second sealable coating 190 can be applied according to a discontinuous coating pattern. Such discontinuous coating pattern may comprise multiple lines, strips, dots or other geometric shapes which may at least partially overlap with the first sealable coating at an inner surface of the bag. Preferably, the first sealable coating 140 has a basis weight between 5 g/m² and 25 g/m², more preferably between 7 g/m² and 20 g/m², and even more preferably between 8 g/m² and 16 g/m².

Before sealing the bag near the top 120′ thereof a stack of absorbent articles 200 is inserted in the bag 100′. The absorbent articles 200 preferably have a rectangular shape as illustrated in FIG. 1 such that the dimensions of the stack of absorbent articles 200 substantially correspond with the dimensions of the available inner space of the bag 100′. In alternative embodiments the absorbent articles 200 and/or the bag 100′ are shaped and/or dimensioned in another manner. It is noted that, for the sake of efficient filling of the bag, it is preferred that the shape and dimensions of the stack of absorbent articles substantially corresponds with the shape and dimensions of the inner space of the bag 100′. More in particular, the width w of the bottom 110 of the bag 100′ should be sufficiently large to accommodate the stack of absorbent articles 200 having a width Wa. In addition, the peripheral wall 130 of the bag 130 should be sufficiently high to accommodate the stack of absorbent articles 200 having a height ha. In addition, the length 1 of the bottom 110 of the bag 100′ should be sufficiently large to accommodate the stack of absorbent articles 200 having a length which is determined by the amount of absorbent articles in the stack and the thickness of each absorbent article 200. Preferably, the absorbent articles are folded absorbent articles having a width Wa between 7 cm and 20 cm, and a height ha between 15 cm and 40 cm. The absorbent articles 200 can be any one of baby diapers, pants, and adult incontinence garments. In the illustrated embodiment the stack of absorbent articles 200 is to be inserted in the bag 100′ such that each absorbent article 200 extends in a substantially upright direction between the bottom 110 and the top 120 of the bag 100′. As illustrated in FIG. 1, the absorbent articles 200 are folded before being inserted in the bag 100′. Preferably each absorbent article 200 is folded along a transversal crotch line of the absorbent article 200 such that a crotch portion 201 of the absorbent article is located at one side, and such that a rear and front end 202, 203 of the absorbent article are located on the opposite side. Preferably, the crotch portion 201 is located at the top 120 of the bag 100′ after insertion, whereas the rear and front end 202, 203 are located at the bottom 110 of the bag 100′ after insertion. In this manner, when a closed package of absorbent article is opened at the closed top 120 thereof, a person can conveniently take out one absorbent article at a time, since one end of the absorbent article, i.e. the crotch portion, corresponds with one absorbent article. In other embodiments wherein the folded absorbent articles are arranged differently in the bag or wherein the bag is opened at a different location, each folded absorbent article would correspond with two ends, due to the absorbent article being folded. In such embodiments it is harder for a person to take out only one absorbent article while keeping the remaining absorbent articles in the stack intact.

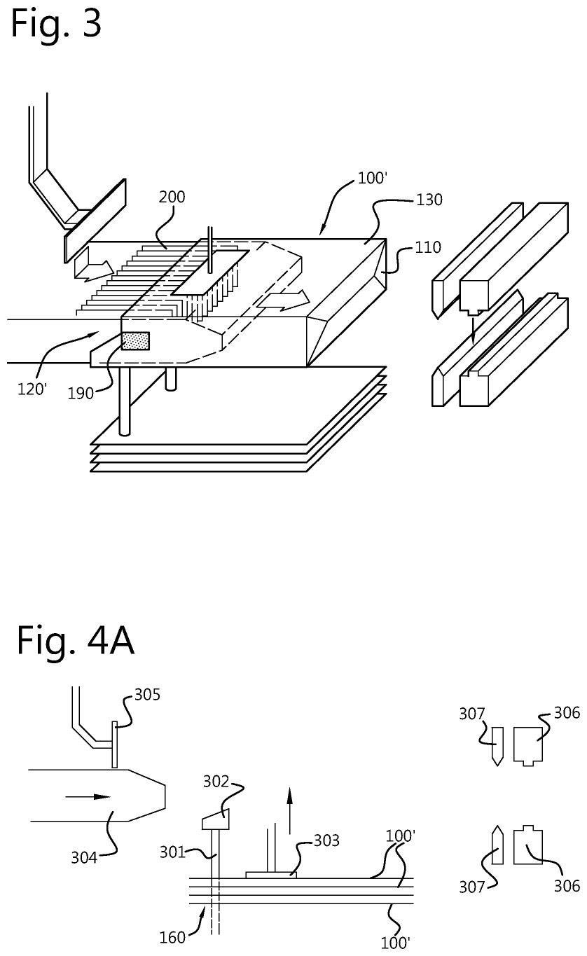

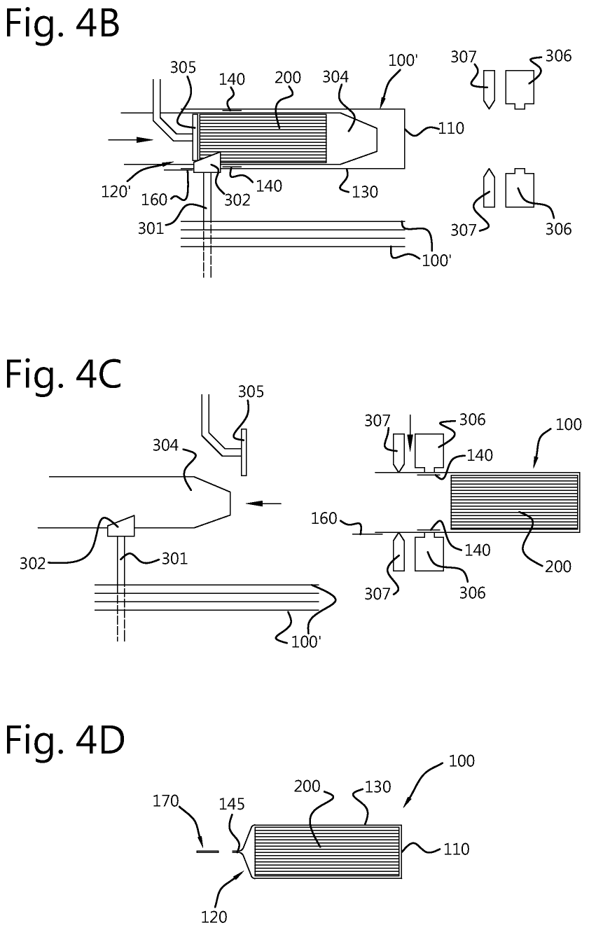

Before the stack of absorbent articles 200 is inserted in the bag 100′ it is preferred that the bag 100′ is adequately opened and is held in an open position until the absorbent articles 200 are inserted in the bag. This is important since non-plastic materials such as paper material or non-woven material, in contrast to plastic materials, are highly susceptible to the risk of being ripped or torn during the filling and manufacturing process of the package. To reduce this risk, the open top end (120′) of the bag is preferably provided with a manipulation part 160 containing a plurality of holes 161. The function of this manipulation part will be further described with reference to FIGS. 3 and 4A-4D.

FIG. 2 illustrates a package 1000 which is formed by arranging the stack of absorbent articles 200 into the open bag 100′ of FIG. 1, and sealing at least a portion 145 of the first sealable coating 140 to obtain a closed top 120. Although not shown in FIG. 2, the second sealable coating 190, i.e. at least a part thereof that overlaps with the sealed portion 145 of the first sealable coating, will cause the second folded portions 122 to be adhered to each other at at least said overlapping part.

As illustrated in FIG. 2 the first side wall 131 is folded inwardly such that two first wing portions or folded portions 121 are formed where the top 120 joins the first side wall 131. The terms “wing portion” and “folded portion” are used interchangeably herein. The two folded portions 121 each comprise, preferably consist of, a sealed portion wherein the first sealable coating is present between two layers of paper material. In the illustrated embodiment, also the second side wall 132 is folded inwardly such that that two second wing portions or folded portions 122 are formed where the top 120 joins the second side wall 132. The two folded portions 122 each comprise, preferably consist of, a sealed portion wherein the first sealable coating is present between two layers of paper material. However, it is noted that embodiments exist wherein only one of the first side wall 131 and second side wall 132 is folded inwardly and wherein consequently only the two first wing portions or folded portions 121 or the two second wing portions or folded portions 122, respectively, are formed. In addition it is noted that the second sealable coating 190 is only provided on the second side wall 132, however it is clear that embodiments exist wherein alternatively, or in addition thereto, the second sealable coating 190 is provided on the first side wall 131. Preferably the second sealable coating 190 is provided on the first side wall 131 and the second side wall 132. The second sealable coating 190 allows for the respective wing portions or folded portions 121, 122, or parts thereof, to be attached or sealed together to form an outer attached portion. Before being attached to each other the two first folded portions 121 together form a substantial V-shape in a top view of the package of absorbent articles, wherein each one of the two first folded portions 121 represents a leg of the V-shape and extends outwardly from a central part of the sealed portion between the first folded portions 121 and the second folded portions 122. After being attached to each other, the two first folded portions 121 together form a substantial line shape in top view of the package of absorbent articles. Before being attached to each other the two second folded portions 122 together form a substantial V-shape in a top view of the package of absorbent articles, wherein each one of the two second folded portions 122 represents a leg of the V-shape and extends outwardly from a central part of the sealed portion between the first folded portions 121 and the second folded portions 122. After being attached to each other, the two second folded portions 122 together form a substantial line shape in top view of the package of absorbent articles. In order to obtain a package of absorbent articles which is easily gripped, preferably, the length ls1 of the attached first folded portions and/or the length ls2 of the attached second folded portions is between 1 cm and 8 cm, more preferably between 2 cm and 7 cm, and even more preferably between 3 cm and 6 cm. In addition, it is preferred that the length ls of the sealed portion between the first folded portions 121 and the second folded portions 122 is between 8 cm and 40 cm, more preferably between 10 cm and 30 cm, and even more preferably between 12 cm and 20 cm.

Preferably an end part 170 of the top 120′ is removed from the bag 100 during or after sealing.

Typically the end part 170 comprises the manipulation part 160. The illustrated manipulation part 160 containing two holes 161 is preferably made of a paper material, e.g. a paper material having a basis weight between 60 and 100 g/m2 measured according to the ISO 536 standard. Alternatively, the manipulation part 160 can be made of a polymer material, e.g. polyethylene material such as a low density polyethylene material. It is noted that the manipulation part 160 can either be a separate part which is attached, e.g. glued, to the bag 100′ or an integral part of the bag. In the illustrated embodiment the manipulation part 160 is substantially rectangular and has a length lm between 20 cm and 35 cm and a width wm between 4 cm and 15 cm. As will be described with reference to FIGS. 3 and 4A-4D the manipulation part 160 is configured to aid in the manufacturing process of the package 1000 and is of little or no use afterwards.

FIG. 3 illustrates a time point in the manufacturing process of the package 1000 of FIG. 2, wherein a bag 100′ is pulled up from a stack of bags by a suction means. In embodiments where the bag 100′ is made of paper material having a basis weight between 60 and 100 g/m², the rigidity of the paper material allows for the bag to be efficiently gripped by the suction means and consequently be pulled up. This is beneficial over the use of plastic material bags as such plastic material would tend to be sucked in by the suction means or at least be severely deformed at the location where the suction means attempt to grip the bag. The bags 100′ are provided with holes 161 which serve as guiding means along a number of corresponding pins for stacking a plurality of bags and pulling up one bag after another. Preferably the holes 161 are provided in a manipulation part which corresponds with an extension on at least one of the front wall, rear wall, first side wall and second side wall. With reference to FIGS. 1 and 2, the manipulation part in FIG. 3 is provided on the rear wall of the bag 100′, which is opposite to the front wall 130. The manipulation part with the plurality of wholes can be a separate part which is attached to the bag 100′ or it may be formed as an integral part of the bag 100′. The holes 161 are configured to firstly serve as a guiding means along the corresponding pins, however, in a later step of the manufacturing process the bag 100′ is pulled or pushed of from the pins towards a sealing means, which pull or push causes the holes 161 to be ripped or torn. In such embodiments it is important that the material surrounding the holes 161 allow for the holes to be ripped open in a controlled manner when needed, and only when needed. The holes 161 should remain intact at least during the steps of pulling up and open the bags, and keeping the open bag in position such that a stack of absorbent articles 200 can be inserted into the bag 100′. Preferably the material surrounding the holes is paper material with a basis weight between 60 and 100 g/m². This can be the same material as the material of the bag or it can be a different material. When paper material is used in the area surrounding the holes 161, i.e. for the manipulation part 160, the holes 161 preferably have a substantially circular circumference. In this manner it is achieved that the holes 161 can properly fulfil their guiding function without being prematurely ripped. In another embodiment a polymer material, e.g. low density polyethylene material, is used in the area surrounding the holes 161, i.e. for the manipulation part. In such embodiment it is preferred that the holes 161 have a substantially cross-shaped circumference. In this manner it is achieved that the holes 161 can be properly pulled open to release the bag 100′ from the pins. The idea is that for materials which are quite rigid and/or are prone to being ripped or torn prematurely, such as paper material, the holes 161 are preferably shaped such that substantially no directional features such as grooves or slits are present in the holes 161. In this manner, it is avoided that a directional force would be exerted on the holes, e.g. by the pins, that would tear open the holes unintentionally. On the other hand, for materials which are quite flexible and/or are difficult to rip or tear to release the bag 100′ from the pins when needed, such as polyethylene material, the holes 161 are preferably shaped such that slit or groove like features are present in the holes 161 wherein the slit or groove like features are at least oriented along an intended tear direction. In this manner, the holes 161 can efficiently be torn open when the bag 100′ is to be released by the pins. Alternatively, the holes 161 may be slid over the pins and/or the pins may briefly be retracted, such that the bag 100′ is transported to the sealing means without the holes being ripped open.

The bags 100′ in the stack have a closed bottom 110 and an open top 120′. During or briefly after being pulled up, the respective bag 100′ is opened up, for example by an air flow, and a stack of absorbent articles 200 is inserted into the bag 100′ via the open top 120′. The stack of absorbent articles 200 is prearranged between two holding means which are configured to insert the prearranged stack of absorbent articles 200 into the bag 100′. During filling of the bag 100′ each of the holding means passes in close proximity to the respective first and second side wall with the stack of absorbent articles 200 arranged there between. A pushing means is configured to push the prearranged stack of absorbent articles 200 from between the holding means, such that the stack of absorbent articles is arranged in the bag 100′ such that each absorbent article 200 extends substantially perpendicular to the closed bottom 110 of the bag 100′. Preferably the pushing means are further configured to push the bag 100′ with the stack of absorbent articles towards a sealing means, thereby ripping the holes 161 and releasing the bag 100′ from the pins. Consequently the open bag 100′ containing the stack of absorbent articles 200 is moved through or passed a sealing means and preferably adjacent cutting means. Preferably a folding means, not shown, is provided which is configured to, before the bag is sealed, fold the first and/or the second side wall inwardly at the top 120′, such that two first wing portions 121 are formed where the top 120 joins the first side wall 131 and/or such that two second wing portions 122 are formed where the top 120 joins the second side wall 132. The sealing means are configured to seal the bag 100′ at the top 120′ thereof by sealing at least a portion of a first sealable coating 140 provided near the top of the bag, preferably on an inner surface of the bag. In the course of sealing a portion of the first sealable coating 140 at the inner surface of the bag, a portion of the second sealable coating 190 which is positioned at the outer surface of the bag, but at the level over the sealed portion 145 of the first sealable coating, will also be sealed resulting in an attachment between two folded portions which are facing each other, thereby realizing at least one outer attached portion. The cutting means are configured to, during or after sealing, cut away an end part of the top 120′ of the bag.

FIGS. 4A-4D illustrate multiple steps of a method for manufacturing a package for a stack of absorbent articles according to an embodiment. FIG. 4A illustrates a stack of bags 100′, wherein each bag is provided with a plurality of holes 161, for example two holes 161. A corresponding number of pins 301, for example two pins 301, extend through the plurality of holes 161. In this manner each bag 100′ in or on the stack has substantially the same orientation. In FIG. 4A each bag 100′ is oriented such that it has an open top 120′ at the left hand side and a closed bottom 110 at the right hand side. In addition the bags 100′ are oriented such that the front wall 133 of the bag is facing up, while the rear wall 134 is located below the front wall 133. In the illustrated embodiment the lower rear wall 134 is extended as compared to the upper front wall 133, because the rear wall 134 is provided with a manipulation part 160 comprising the plurality of holes 161. In other words, the lower rear wall 134 is longer as compared to the upper front wall 133. A suction means 303 is illustrated which is configured to engage on the front wall 133 of the upper bag 100′ of the stack, and pull up the upper bag 100′. While doing so, the rear wall 134 is pulled up as well and the holes 161 of the manipulation part 160 are pulled up along the pins 301 until they reach a respective stopper element 302, located at a top of each pin 301. In this manner, the rear wall 134 comprising the manipulation part 160 remains at the level of the stopper elements 302 while the front wall 133 is pulled up further, thereby opening the bag 100′ as is further illustrated in FIG. 4B. The functionality of the holding means 304, pushing means 305, sealing means 306 and cutting means 307 will be further elaborated below.

FIG. 4B illustrates the upper bag 100′ in its pulled up and open state. A first sealable coating 140 is present on the bag near the top 120′. The first sealable coating 140 is located on the front wall 133 and/or the rear wall 134. Preferably the first sealable coating 140 is located on an inner surface of the front wall 133 and/or rear wall 134. A stack of absorbent articles 200 is provided, in this case by holding means 304, and inserted in the opened bag 100′ via open top 120′. Pushing means 305 comprises a pushing surface which is dimensioned in such a way that it fits in between the holding means 304 and which substantially corresponds with the surface of the opening at the open top 120′ and/or with the surface of the closed bottom 110. The pushing surface is preferably rectangular. The pushing means 305 are configured to push the stack of absorbent articles 200 from between the holding means 304 towards the closed bottom 110 of the bag 100′. When the stack of absorbent articles 200 comes into contact with the closed bottom 110, the pushing means 305 further cause the bag 100′ and the stack of absorbent articles 200 arranged therein to be moved towards a sealing means 306. Preferably, also a cutting means 307 is provided. This will be further elaborated in view of FIG. 4C and 4D.

FIG. 4C illustrates the bag 100′ in a pushed through position, ready to be sealed. In this position at least a portion of the first sealable coating 140 is located in between the sealing means 306. The portion 145 of the first sealable coating 140 is then sealed by means of pressure and/or heat such that a closed top end 120 of the bag 100 is achieved. Preferably, the first sealable coating 140 is a pre-coating which is applied to the bag in advance, and which is dry when the stack of absorbent articles are inserted into the bag 100. Preferably, before the step of sealing, the first and the second side wall are folded inwardly at the top 120, such that two first wing portions 121 are formed where the top 120 joins the first side wall 131 and such that two second wing portions 122 are formed where the top 120 joins the second side wall 132. These wing portions 121, 122 are illustrated in FIGS. 2, 5 and 6 and are described in the corresponding passages of this description. Preferably, during or after the step of sealing the bag, an end part 170 of the top 120′ of the bag is cut off. For example, the cut of end part 170 may comprise the manipulation part 160 or at least a part thereof comprising the holes 161 which may or may not be ripped open at that moment in the manufacturing process, depending on the respective embodiment. Preferably the cut is executed along a cutting line which is at least partially serrated. In this manner a serrated portion remains near de sealed top of the package, which serrated portion allows for a user to easily tear open the package. Alternatively or in addition the cutting line may comprise any one of a substantially straight line, waved line, ribbed line or a combination thereof. FIG. 4D schematically illustrates a closed bag 100 with a stack of absorbent articles 200 arranged therein and a cut off end part 170. In the final package the stack of absorbent articles 200 is arranged in the bag 100 such that each absorbent article extends substantially perpendicular to the closed bottom 110 of the bag 100.

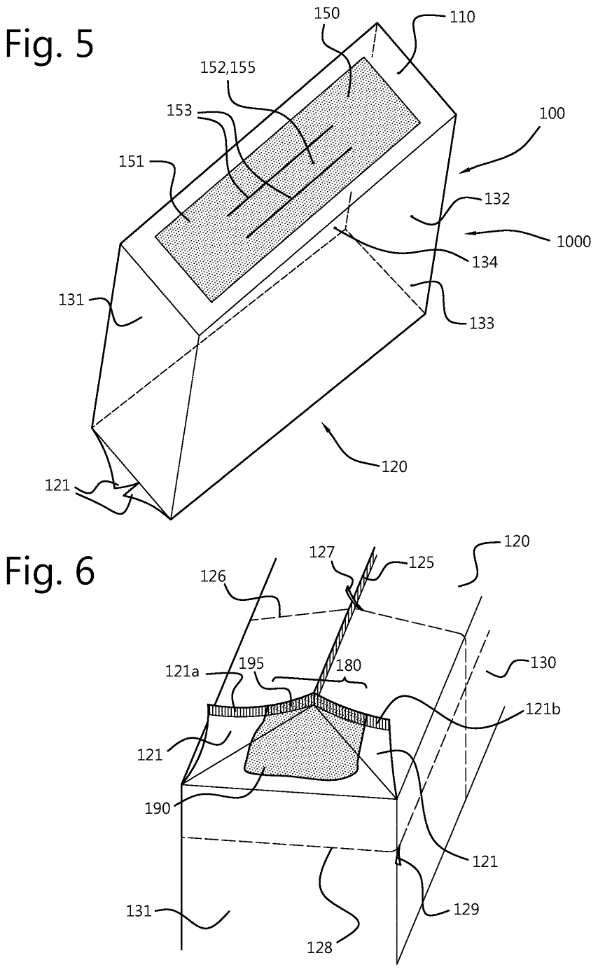

Preferably, the closed bottom is formed by folding and gluing a portion of the paper material, such that is has a substantially rectangular shape. It can be seen that at least at the peripheral wall 130 and the closed bottom 110 the bag 100 is tightly arranged against the stack of absorbent articles 200. At the top 120 of the package a free volume is present in the bag between the stack of absorbent articles 200 and the sealed portion 145. On the one hand, the free volume is provided as a buffer such as to create a safety distance between the absorbent articles and the sealable top 120 of the bag to avoid that the absorbent articles in the bag are affected by the sealing means 306 when sealing the bag 100. On the other hand, the free volume forms a depressible, collapsible area at the outside of the bag 100. This area can be more easily gripped by users or caregivers as compared to the tightly wrapped peripheral wall 130 and bottom 110. Further, the sealed portion of the top 120 can be seen as to protrude from the bag 100 and thereby provides an additional gripping area for a user or caregiver. In further preferred embodiment even more easily graspable areas are provided at the outside surface of the bag 100 as further elaborated in view of FIGS. 5 and 6.

FIG. 5 illustrates a package 1000 according to a further embodiment wherein the bottom 110 is provided with a handle 155. The handle 155 is illustrated as being substantially aligned with a length direction of the bottom 110 but it is clear that embodiments exist wherein the handle 155 is oriented in another direction. In this manner additional gripping means are provided on an otherwise hard to grasp area of the package 1000. Preferably the handle is made of non-plastic material such as paper material. In the illustrated embodiment the package 1000 comprises a paper sheet 150 with two cuts 153. The paper sheet is attached to the bottom 120 of the bag in an area 151 around the two cuts, such that an area 152 of the paper sheet between the two cuts functions as the handle 155.

FIG. 6 illustrates a further embodiment of a package, which is provided with a number of features which allow easy opening of the non-plastic bag. Non-plastic bags and especially paper bags are harder to open as compared to thin plastic bags which can be gripped at a random location and can be easily torn open at that location. FIG. 6 shows that at the top 120 of the bag a seal seam 125 is formed. In addition to the seal seam 125, two first wing portions or folded portions 121 are formed where the top 120 joins the first side wall 131. As described earlier, these wing portions or folded portions 121 are formed by folding the first side wall 131 inwardly before sealing the top 120 of the bag. In addition to acting as gripping means, these wing portions 121 respectively comprise wing portion seam 121a and wing portion seam 121b which allow for an easy opening of the bag. More in particular, the wing portions 121 and their corresponding seams 121a, 121b are dimensioned and formed in such a way, that the separation force needed to separate the seams 121a, 121b is substantially smaller as compared to the separation force needed to separate the seal seam 125. To even further customize the functionality and/or appearance of the bag a second sealable coating 190 is provided which allows for at least a part of the wing portion seams 121a, 121b and optionally the entire wing portions seams 121a, 121b to be adhered or sealed to each other. This seal will be performed at least at a sealed portion 195 where the second sealable coating at the outer surface of the bag overlaps with the sealed portions 121a, 121b on the inner surface of the bag. In this manner a visual continuation of the seal seam 125 can be formed by realizing an outer attached portion 180(please note that this is not illustrated in FIG. 6.) Preferably the end part 170 which is cut of from the top 120 during or after sealing, is cut of such that a ribbed or serrated seam portion remains at the top 120 of the bag. In this manner the seal seam 125 and/or wing portion seams 121a, 121b comprise a ribbed or serrated portion which can be easily torn by applying a tearing force transversally on the seal seam 125 and/or wing portion seams 121a, 121b. In addition or alternatively, the bag may be provided with one or more tear strips 126, 128 to enable easy opening of the bag. A tear strip 126, 128 may be attached or incorporated in one or more of the walls of the paper bag and preferably comprise a tear line opener 127, 129 which at least partially extends or protrudes from the respective wall of the paper bag. The tear strip 126, 128 and/or tear line opener 127, 129 may be formed out of cotton, polypropylene or a combination thereof. In the illustrated embodiment tear strip 128 is positioned in such a way as to cause the package to be opened along a plane which is substantially parallel to the top and/or bottom of the package. The tear strip 128, optionally in combination with a corresponding tear line opener 129, allows for the top of the package to be at least partially removed, such that a person can easily take out an absorbent article from above, especially when the absorbent articles are arranged in the bag according to one of the previously described preferred embodiments. In addition or alternatively, a tear strip 126 can be provided which is positioned in such a way as to cause the package to be opened along a plane which is substantially perpendicular to the top and/or bottom of the package. The tear strip 126, optionally in combination with a corresponding tear line opener 127, allows for a sidewall of the package to be at least partially removed, such that a person can easily take out an absorbent article from the side, especially when the opened package is stored or placed on its opposite side. It is clear to the skilled person that other tear strips may be provided which are positioned and/or oriented differently in addition to or as alternative to the illustrated tear strips 126 and/or 128. It is noted that any one of the plurality of features illustrated in FIG. 6 can be applied in isolation or in combination with any other illustrated feature.

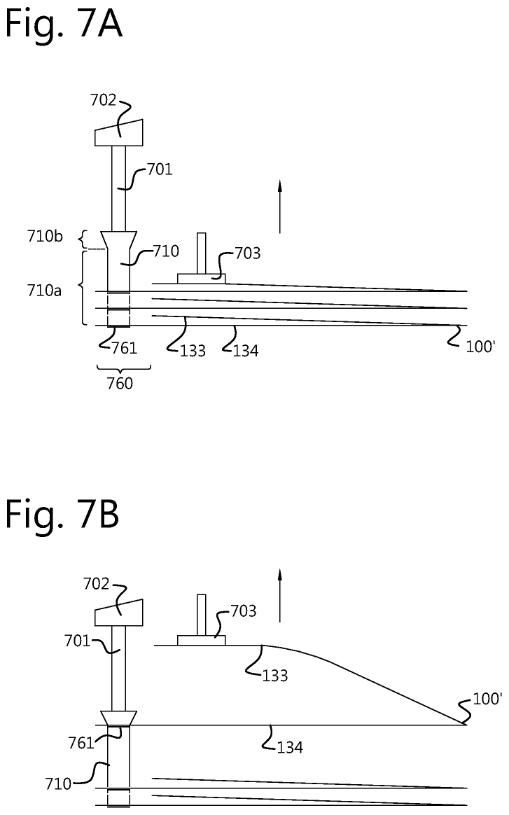

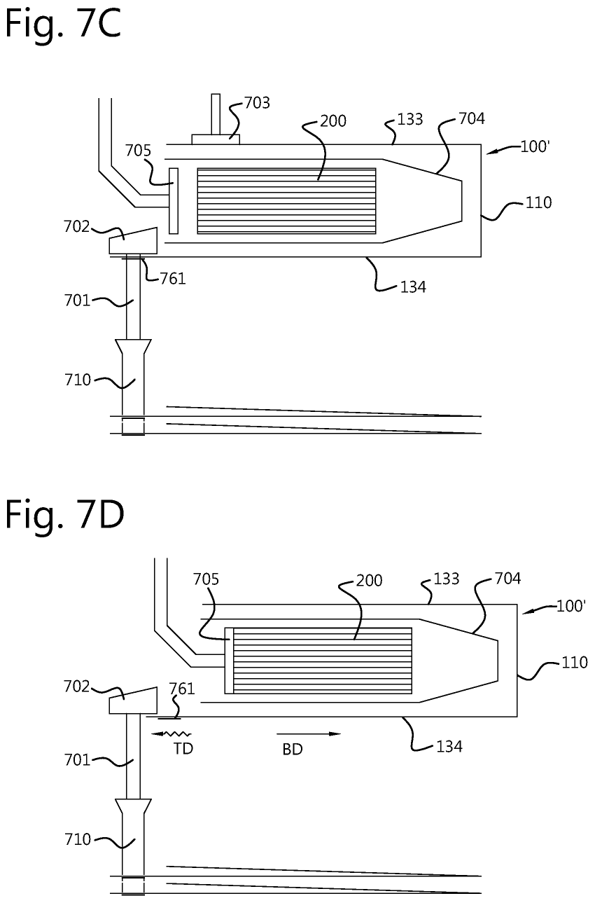

FIGS. 7A-7D illustrate multiple steps of a method for manufacturing a package for a stack of absorbent articles according to an embodiment wherein the plurality of pins 701 is provided with a plurality of retarding elements 710. In FIG. 7A a bag 100′ is to be pulled up from a stack of bags by a suction means 703. The bags 100′ are illustrated as folded bags wherein the front wall 133 is facing upwards and the rear wall 134 is facing downwards, and wherein the rear wall is provided with a manipulation part 760 comprising a plurality of holes 761. It is clear to the skilled person that this is one of various possible orientations of the bag and by extension of the stack of bags and that other orientations are possible as well. In addition or alternatively, the manipulation part 760 may be provided on at least one of the front wall, first side wall and second side wall. The manipulation part 760 with the plurality of holes 761 may be a separate part which is attached to the bag 100′ or it may be formed as an integral part of the bag 100′. The holes 761 serve as guiding means along a number of corresponding pins 701 for stacking a plurality of bags and pulling up one bag after another. The pins 701 are preferably provided with a plurality of retarding elements 710. The holes 761 are configured to firstly serve as a guiding means along the corresponding pins and retarding elements. However, in a later step of the manufacturing process the bag 100′ is pulled or pushed of from the pins 701 towards a sealing means, which pull or push causes the holes 761 to be ripped or torn as illustrated in FIG. 7D. It is beneficial that the holes are ripped open in a controlled manner when needed, and only when needed. Preferably, the holes 761 remain at least partially intact during the steps of pulling up and opening the bags, and keeping the open bags in position such that a stack of absorbent articles 200 can be inserted into the bag 100′. Preferably the material surrounding the holes 761 is paper material with a basis weight between 60 and 100 g/m². This can be the same material as the material of the bag or it can be a different material.

In FIG. 7A each bag 100′ is oriented such that it has an open top at the left hand side and a closed bottom 110 at the right hand side. At the open top the lower rear wall 734 is extended as compared to the upper front wall 733, by providing the manipulation part 760 comprising the plurality of holes 761. The bags are positioned such that a pin 701 comprising a retarding element 710 extends through each hole 761. Preferably the retarding element 710 is positioned at a bottom of the pin 701 such that the retarding element 710 extends through the hole 761 when the bag 100′ is on the stack. A suction means 303 is illustrated which is configured to engage on the front wall 133 of the upper bag 100′ of the stack, and pull up the upper bag 100′. While doing so, the rear wall 134 is pulled up as well and the holes 761 of the manipulation part 760 are pulled up along the retarding elements 710. The retarding element 710 is configured to retard or slow down the upward movement of the rear wall 134 relative to the front wall 133, such that the bag 100′ starts to open while the bag 100′ is pulled up as is illustrated in FIG. 7B.

At the time of FIG. 7B the bag is partially pulled over the retarding element 710. More in particular, the front wall 133 is pulled up beyond the retarding element, whereas the rear wall 134 is positioned further down. The illustrated retarding element 710 comprises a first portion 710a and a second portion 710b. The first portion 710a is substantially cylindrical and the second portion 710b is substantially funnel shaped. However, other configurations of the retarding element 710 are possible in order to achieve the intended retarding effect. The retarding element 710 surrounds at least part of the corresponding pin 301. Preferably the pin 301 and the retarding element 710 are concentric. While being pulled up along the retarding element 710, the respective hole 761, more in particular a circumference thereof, will be subject to some friction and/or resistance. This will cause the manipulation part 760 and the rear wall 134 to be delayed during the upward movement. Consequently, since the front wall 133 does not encounter such friction and/or resistance, the front wall 133 is pulled up faster and the bag is at least partially opened.

In FIG. 7C the bag is completely pulled over the retarding element 710 and the corresponding pin 701 and is in a further opened state. The holes 761 have encountered the stopping element 702 which avoids that the rear wall 134 with the manipulation part 760 is pulled further up. At this moment a stack of absorbent articles 200 is provided by holding means 704, and inserted in the opened bag 100′ via open top 120′. In addition pushing means 705 are provided which are configured to push the stack of absorbent articles 200 from between the holding means 704 towards the closed bottom 110 of the bag 100′. The pushing means 705 further cause the bag 100′ and the stack of absorbent articles 200 arranged therein to be moved towards a sealing means at the right hand side of the figure (not illustrated).

As can be seen in FIG. 7D the bag 100′ is moved in a bag direction (BD), away from the pins 701. Consequently the manipulation part 760 is torn from the pins 701 causing a tear extending from the holes 761 in a tear direction (TD), which is substantially opposite to the bag direction.

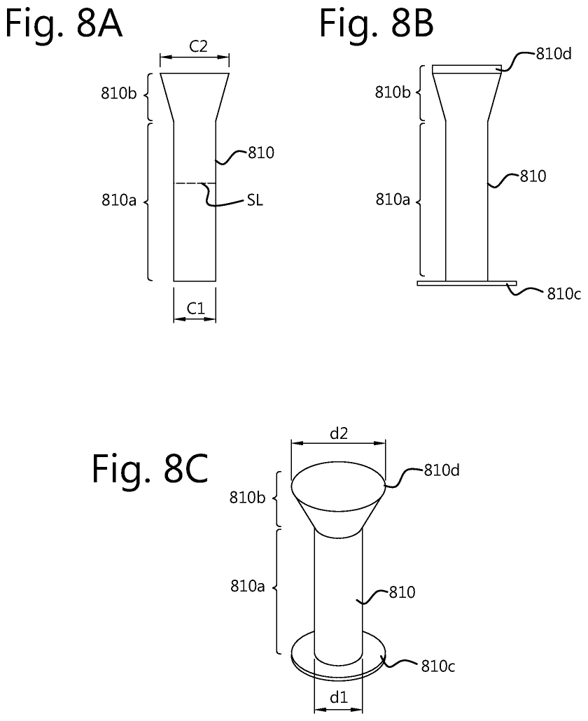

In the course of the different steps as illustrated in FIGS. 7A-7D, the holes 761 are guided along the retarding elements 710 and the pins 701, before being ripped from the pins 701. On the one hand, the holes 761 maintain their integrity to fulfil their guiding function during the first steps of this process, and, on the other hand, the holes 761 are easily torn of the pins 701 in a controlled manner at the end of this process. In general, it is clear that it is beneficial that the shape and dimensions of the holes 761 substantially correspond with the shape and dimensions of the retarding elements 710, especially in view of cross-section and/or circumference. For example, if the pins 701 and retarding elements 710 have a substantially circular cross-section, the holes 761 preferably have a substantially circular circumference, with suitable dimensions such that the holes 761 can be efficiently guided along the pins 701 and/or retarding elements 710 with a low risk of premature tearing. A similar reasoning is applicable for other shapes such as ovals, polygons, etc. In addition, it has been found that particular configurations of the retarding elements 710 and/or holes 761 offer further benefits, as will be further elaborated in view of FIGS. 8A-8C and 9A-9G. It will be clear that any one of the configurations and features as elaborated below can be either applied in isolation or can be mutually combined.

FIGS. 8A-8C illustrate exemplary embodiments of retarding elements. The retarding elements are to be positioned along at least part of the pins. Preferably they are positioned at a bottom part of the pins as illustrated in FIGS. 7A-7D. Alternatively or in addition the pins are positioned at a middle part and/or top part of the pins and/or extend along substantially the entire length of the pins.

FIG. 8A illustrates a cross-section view of a retarding element 810 comprising a first portion 810a and a second portion 810b. The first portion 810a is positioned under the second portion 810b. The first portion 810a is preferably substantially cylindrical and has a cross-section distance C1. In case of e.g. an oval shape or a polygonal shape, the first cross-section distance may correspond with a maximum cross-section distance. The second portion 810b is substantially preferably funnel shaped and has a first cross-section distance C1 and a second cross-section distance C2, wherein C2 is larger than C1. In a preferred embodiment, the first portion 810a is a cylindrical portion having a first diameter d1 (see FIG. 8C), and the second portion is a conical portion 810b having a diameter which increases from the first diameter d1 to a second larger diameter d2, and the manipulation part is first guided along the first portion 810a and next along the second portion 810b. More preferably, the plurality of holes 761, 791 (see FIGS. 7A-7D and FIGS. 9A-9G discussed below) comprises a plurality of round holes having a diameter dh between the first diameter d1 and the second diameter d2, i.e. d1<dh<d2. If the diameter of the hole would be smaller than d1, there is a severe risk of premature ripping of the hole. If the diameter of the hole would be larger than d2 there would be too much space between the retarding element 810 and the circumference of the hole. This would cause the bag not to be properly guided and/or the manipulation part comprising the hole not to be properly retarded. To further improve guiding, retarding and/or tearing functionality the holes are preferably provided with tear inducing elements, as will be further elaborated in view of FIGS. 9A-9G.

Preferably the retarding element 810 comprises two parts, which can be separated from each other as indicated by the dashed separation line SL in FIG. 8A. In this manner, a part of the retarding element having the maximal cross-section distance C2 can be temporarily removed, such that a stack of bags, and more in particular the manipulation parts of the bags with the plurality of holes, can easily be placed over the remaining part of the retarding element having the minimal cross-section distance. The second portion 810b of the retarding element 810 is illustrated as a funnel wherein the cross-section distance gradually increases. In this manner the risk of premature ripping of the hole is reduced. Preferably the retarding element is made of polyethylene, more preferably high-density polyethylene. Alternatively or in addition, other materials having similar mechanical and/or frictional properties can be used.

FIG. 8B illustrates a cross-section view of a further embodiment of a retarding element 810 further comprising a third portion 810c and a fourth portion 810d. The third portion 810c corresponds with a platform portion of the retarding element and is substantially disc shaped. The third portion 810c has a cross-section distance which is significantly larger than C2. The third portion 810c increases the stability of the retarding element 810 and/or ensures that bags which are stacked on the retarding element 810 remain in position. The fourth portion 810d corresponds with a top portion of the retarding element 810 and has a cross-section distance which is substantially equal to C2. The function of the fourth portion 810d is to further improve guiding and/or retarding functionality of the retarding element.

FIG. 8C is a perspective view of the retarding element 810 of FIG. 8B, illustrating the preferred cylindrical shape of the first portion 810a and the preferred funnel shape of the second portion 810b.

### Glossary

As used in the present application, the following terms have the following meanings:

“A”, “an”, and “the” as used herein refers to both singular and plural referents unless the context clearly dictates otherwise. By way of example, “an edge barrier” refers to one or more than one edge barrier.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or variations are appropriate to perform in the disclosed invention. However, it is to be understood that the value to which the modifier “about” refers is itself also specifically disclosed.

“Absorbent article”, “absorbent garment”, “absorbent product”, “absorbing article”, “absorbing garment”, “absorbing product” and the like as used herein are used interchangeably and refer to devices that absorb and contain bodily exudates, and more specifically, refers to devices that are placed against or in proximity to the body of the wearer to absorb and contain the various liquids discharged from the body. Absorbent articles include but are not limited to feminine hygiene garments, baby diapers and pants, adult incontinence garments, various diaper and pants holders, liners, towels, absorbent inserts and the like.

“Absorbent core” as used herein refers to a three-dimensional part of the absorbent structure, comprising liquid-absorbing material, useful to permanently absorb and/or retain bodily exudates.

“Absorbent component” as used herein refers to a structural constituent of an absorbent article, e.g., a piece of an absorbent core, such as one of multiple pieces in a multi-piece absorbent core.

“Absorbent element” as used herein refers to a part of a functional constituent of an absorbent structure, e.g., an acquisition layer, a dispersion layer, core layer or a release structure formed of a material or materials having particular liquid handling characteristics suitable for the specific function.

“Absorbent fibrous polymer material” as used herein refers to an absorbent polymer material which is in threadlike from such as fibers, filaments, and the like so as to be less flowable in the dry state than particulates.

“Absorbent insert” as used herein refers to a device adapted for insertion into an “Absorbent layer” as used herein refers to a term referring to a discrete, identifiable sheet-like or web-like element of an absorbent article which may remain detached and relatively movable with respect to another such element or may be attached or joined so as to remain permanently associated with another such element. Each absorbent layer may itself include a laminate or combination of several layers, sheets and/or webs of similar or diverse compositions.

“Absorbent polymer material”, “absorbent gelling material”, “AGM”, “superabsorbent”, “superabsorbent material”, “superabsorbent polymer”, “SAP” and the like as used herein are used interchangeably and refer to any suitable particulate (e.g., flaked, particulate, granular, or powdered) or fibrous cross linked polymeric materials that can absorb at least 5 times and preferably at least about 10 times or more its weight of an aqueous 0.9% saline solution as measured using the Centrifuge Retention Capacity test (EDANA 441.2-01).

“Absorbent polymer material area” as used herein refers to the area of the absorbent structure wherein adjacent layers are separated by a multiplicity of absorbent polymer material. Incidental contact areas between these adjacent layers within the absorbent particulate polymer material area may be intentional (e. g bond area's) or unintentional (e.g. manufacturing artifacts). “Absorbent particulate polymer material” as used herein refers to an absorbent polymer material which is in particulate form such as powders, granules, flakes and the like so as to be flowable in the dry state.

“Absorption” as used herein refers to the process by which a liquid is taken up within a material.

“Absorption rate” as used herein refers to the rate of absorption of liquid, i.e. the amount of liquid which is absorbed per unit of time, typically by an absorbent component, element and/or absorbent layer of the absorbent article, structure and/or core.

“Acquisition layer”, “acquisition region”, “acquisition surface” or “acquisition material” and the like as used herein refer to the layer overlying the absorbent core having a faster liquid uptake and/or distribution capability.

“Absorbency” is the ability of a material to take up fluids by various means including capillary, osmotic, solvent, chemical and/or other action.

“Adult incontinence garment” as used herein refers to absorbent articles intended to be worn by incontinent adults, for absorbing and containing bodily exudates.

“Adhesion” as used herein refers to the force that holds different materials together at their interface.

“Adhesive” as used herein refers to a material, which may or may not be flowable in solution or when heated, that is used to bond materials together.

“Adsorption” as used herein refers to the process by which a liquid is taken up by the surface of a material.

“Airlaying” as used herein refers to forming a web by dispersing fibers or particles in an air stream and condensing them from the air stream onto a moving screen by means of a pressure and/or vacuum; a web of fibers produced by airlaying is herein referred to an “airlaid”; an airlaid web bonded by one or more techniques to provide fabric integrity is herein referred to an “airlaid nonwoven”.

“Apparent density”, “density” as used herein refers to the basis weight of the sample divided by the caliper with appropriate unit conversions incorporated therein. Apparent density used herein has the unit g/cm3.

“Attach”, “attached” and “attachment” as used herein are synonymous with their counterparts of the terms “fasten”, “affix”, “secure”, “bind”, “join” and “link”.

“Baby diaper” as used herein refers to absorbent articles intended to be worn by children, for absorbing and containing bodily exudates which the user draws up between the legs and fastens about the waist of the wearer.

“Baby pants” as used herein refers to absorbent articles marketed for use in transitioning children from diapers to underwear intended to cover the lower torso of children, so as to absorb and contain body exudates which article is generally configured like a panty garment and manufactured with a completed waist encircling portion, thereby eliminating the need for the user to fasten the article about the waist of the wearer.

“Back region” as used herein refers to the portion of an absorbent article or part thereof that is intended to be positioned proximate the back of a wearer.

“Backing” as used herein refers to a web or other material that supports and reinforces the back of a product.

“Basis weight” is the weight per unit area of a sample reported in grams per square meter, g/m2 or gsm.

“Bodily exudates”, “body exudates”, “bodily fluids”, “body fluids”, “bodily discharges”, “body discharges”, “fluid(s)”, “ liquid(s)”, “fluid(s) and liquid(s) and the like as used herein are used interchangeably and refer to, but are not limited to urine, blood, vaginal discharges, breast milk, sweats and fecal matter.

“Binder”, “adhesive”, “glue”, “resins”, “plastics” and the like as used herein are used interchangeably and refer to substances, generally in a solid form (e.g. powder, film, fiber) or as a foam, or in a liquid form (e .g. emulsion, dispersion, solution) used for example by way of impregnation, spraying, printing, foam application and the like used for attaching or bonding functional and/or structural components, elements and materials, for example including heat and/or pressure sensitive adhesives, hot-melts, heat activated adhesives, thermoplastic materials, chemical activated adhesives/solvents, curable materials and the like.

“Bond strength” as used herein refers to the amount of adhesion between bonded surfaces. It is a measure of the stress required to separate a layer of material from the base to which it is bonded.

“Capillary action”, “capillarity”, or “capillary motion” and the like as used herein are used to refer to the phenomena of the flow of liquid through porous media.

“Chassis” as used herein refers to a foundational constituent of an absorbent article upon which the remainder of the structure of the article is built up or overlaid, e.g., in a diaper, the structural elements that give the diaper the form of briefs or pants when configured for wearing, such as a backsheet, a topsheet, or a combination of a topsheet and a backsheet.

“Cellulose fibers” as used herein refers to naturally occurring fibers based on cellulose, such as, for example cotton, linen, etc; wood pulp fibers are one example of cellulose fibers; man-made fibers derived from cellulose, such as regenerated cellulose (rayon), or partially or fully acetylated cellulose derivatives (e.g. cellulose acetate or triacetate) are also considered as cellulose fibers.

“Cluster” or the like as used herein refers to an agglomeration of particles and/or fibers.

“Chemically stiffened fibers”, chemically modified fibers”, “chemically cross-linked fibers”, “curly fibers” and the like as used herein are used interchangeably and refer to any fibers which have been stiffened by chemical means to increase stiffness of the fibers under both dry and aqueous conditions, for example by way of addition of chemical stiffening agents (e.g. by coating, impregnating, etc), altering the chemical structure of the fibers themselves (e.g. by cross-linking polymer chains, etc) and the like.

“Cohesion” as used herein refers to the resistance of similar materials to be separated from each other.

“Compartment” as used herein refers to chambers, cavities, pockets and the like.

“Comprise,” “comprising,” and “comprises” and “comprised of” as used herein are synonymous with “include”, “including”, “includes” or “contain”, “containing”, “contains” and are inclusive or open-ended terms that specify the presence of what follows e.g. a component and do not exclude or preclude the presence of additional, non-recited components, features, elements, members, steps, known in the art or disclosed therein.

“Coverstock” as used herein refers to a lightweight non-woven material used to contain and conceal an underlying absorbent core material; examples are the facing layer or materials that cover the absorbent cores of feminine hygiene garment s, baby diapers and pants and adult incontinence garments.

“Crotch region” of an absorbent article as used herein refers to about 50% of the absorbent article's total length (i.e., in the y-dimension), where the crotch point is located in the longitudinal center of the crotch region. That is, the crotch region is determined by first locating the crotch point of the absorbent article, and then measuring forward and backward a distance of 25% of the absorbent article's total length.

“Cross direction (CD)”, “lateral” or “transverse” and the like as used herein are used interchangeably and refer to a direction which is orthogonal to the longitudinal direction and includes directions within ±45° of the transversal direction.

“Curing” as used herein refers to a process by which resins, binders or plastics are set into or onto fabrics, usually by heating, to cause them to stay in place; the setting may occur by removing solvent or by cross-linking so as to make them in soluble.

“Diaper”, “conventional diaper”, “diaper-like”, “diaper-like garment” and the like as used herein are used interchangeably and refer to disposable absorbent articles, which typically include a front waist portion and a back waist portion which may be releasable connected about the hips of the wearer during use by conventional fasteners such as adhesive tape fasteners or hook and loop type fasteners. In use, the article is positioned between the legs of the wearer and the fasteners are releasable attached to secure the back waist portion to the front waist portion of the diaper, thereby securing the diaper about the waist of the wearer. The front waist portion and a back waist portion are connected by relatively non-stretchable or stretchable members (the term “stretchable” as used herein refers to materials that are extensible when forces are applied to the material, and offer some resistance to extension). Hence, such articles are generally not configured to be pulled up or down over the hips of the wearer when the fasteners are attached.

“Dispersion layer”, “dispersion region”, “dispersion surface” or “dispersion material” and the like as used herein refer to the layer overlying the absorbent core having a faster liquid uptake and dispersion capability.

“Disposable” is used herein to describe articles that are generally not intended to be laundered or otherwise restored or reused (i.e., they are intended to be discarded after a single use and, preferably, to be recycled, composted or otherwise disposed of in an environmentally compatible manner).

“Drylaying” as used herein refers to a process for making a nonwoven web from dry fiber; these terms apply to the formation of carded webs, as well as to the air laying formation of random webs; a web of fibers produced by drylaying is herein referred to as a “drylaid”; a drylaid web bonded by one or more techniques to provide fabric integrity is herein referred to a “drylaid nonwoven”.

“Dry strength” as used herein refers to the strength of ajoint determined in dry state conditions, immediately after drying under specified conditions or after a period of conditioning in the standard laboratory atmosphere.

“Essentially cellulose free”, “substantially fluffless” or “little to no cellulose fibers” as used herein refers to an absorbent article, structure, core component and/or element containing less than 20% by weight cellulosic fibers, less than 10% cellulosic fibers, less than 5% cellulosic fibers, no cellulosic fibers, or no more than an immaterial amount of cellulosic fibers which do not materially affect the thinness, flexibility or absorbency thereof.

“Essentially fluffless” or “little to no fluff pulp” as used herein refers to an absorbent article, structure, core, component and/or element containing less than 20% by weight fluff pulp, less than 10% fluff pulp, less than 5% fluff pulp, no fluff pulp, or no more than an immaterial amount of fluff pulp which do not materially affect the thinness, flexibility or absorbency thereof.

“Fabric” as used herein refers to a sheet structure made from fibers, filaments and/or yarns.

“Feminine hygiene garments” as used herein refer to absorbent hygiene articles intended to be worn by woman, for absorbing and containing body exudates.

“Fiber” as used herein refers to the basic threadlike structure from which nonwovens, yarns and textiles are made. It differs from a particle by having a length at least 4 times its width; “Natural fibers” are either of animal (wool, silk), vegetable (cotton, flax, jute) or mineral (asbestos) origin, while “Man-made fibers” may be either polymers synthesized from chemical compounds (polyester, polypropylene, nylon, acrylic etc.) or modified natural polymers (rayon, acetate) or mineral (glass). “Fiber” and “filament” are used interchangeably.

“Fluff pulp” or “Pulp fluff” as used herein refers to wood pulp specially prepared to be drylaid. The fibers can be either natural or synthetic or a combination thereof.

“Front region” as used herein refers to the portion of an absorbent article or part thereof that is intended to be positioned proximate the front of a wearer.

“Garment facing layer” as used herein refers to elements of the chassis that form the outer surface of the absorbent article, such as the backsheet, the side panels, the waist fasteners, and the like, when such elements are present.

“Heat activated adhesive” as used herein refers to a dry adhesive that is rendered tacky or fluid by application of heat or heat and pressure to the assembly.

“Heat sealing adhesive” as used herein refers to a thermoplastic adhesive which is melted between the adherent surfaces by heat application to one or both of the adjacent adherent surfaces.

“High loft” as used herein refers to general term of low density, thick or bulky fabrics.

“Hot-melt adhesive” as used herein refers to a solid material that melts quickly upon heating, then sets to a firm bond upon cooling; used for almost instantaneous bonding.

“Hydrophilic” as used herein refers to having an affinity for being wetted by water or for absorbing water.

“Hydrophobic” as used herein refers to lacking the affinity for being wetted by water or for absorbing water.

“Immobilization layer” as used herein refers to a layer able to be applied to the absorbent polymer material or absorbent polymer material area with the intent to gather, bond and/or immobilize absorbent material and/or absorbent layer.

“Join”, “joined” and “joining” as used herein refers to encompassing configurations wherein an element is directly secured to another element by affixing the element directly to the other element, as well as configurations wherein the element is indirectly secured to the other element by affixing the element to an intermediate member or members which in turn is or are affixed to the other element.

“Knitting” as used herein refers to the technique for interlocking loops of fibers with needles or similar devices.

“Layer” refers to identifiable components of the absorbent article, and any part referred to as a “layer” may actually comprise a laminate or combination of several sheets or webs of the requisite type of materials. As used herein, the term “layer” includes the terms “layers” and “layered.”

“Upper” refers to the layer of the absorbent article which is nearest to and/or faces the wearer facing layer; conversely, the term “lower” refers to the layer of the absorbent article which is nearest to and/or faces the garment facing layer. “Layer” is three dimensional structure with a x dimension width, y dimension length, and z-dimensions thickness or caliper, said x-y dimensions being substantially in the plane of the article, however it should be noted that the various members, layers, and structures of absorbent articles according to the present invention may or may not be generally planar in nature, and may be shaped or profiled in any desired configuration.

“Machine direction (MD)”, “longitudinal” and the like as used herein are used interchangeably and refer to a direction running parallel to the maximum linear dimension of the structure and includes directions within ±45° of the longitudinal direction.

“Major surface” as used herein refers to a term used to describe the surfaces of greatest extent of a generally planar or sheet-like structural element and to distinguish these surfaces from the minor surfaces of the end edges and the side edges, i.e., in an element having a length, a width, and a thickness, the thickness being the smallest of the three dimensions, the major surfaces are those defined by the length and the width and thus having the greatest extent.

“Mass flow” as used herein refers to the flow of a liquid from one absorbent element or component to another absorbent element or component by channel flow action.

“Mechanical bonding” as used herein refers to a method of bonding fibers by entangling them. This can be achieved by needling, stitching with fibers or by the use of high-pressure air or water jets and the like.

“Nonwoven” as used herein refers to manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as melt blowing, spun bonding, solvent spinning, electrospinning, and carding. The basis weight of nonwoven fabrics is usually expressed in grams per square meter (gsm).

“Pant”, “training pant”, “closed diapers”, “prefastened diapers”, “pull-on diapers” and “diaper-pants” and the like as used herein are used interchangeably and refer to absorbent articles which are typically applied to the wearer by first leading the feet into the respective leg openings and subsequently pulling the pants from the feet to waist area over the hips and buttocks of the wearer and which are capable of being pulled up or down over the hips of the wearer. Typically, such articles may include a front waist portion and a back waist portion which may be connected about the hips of the wearer by integral or releasable members. A pant may be preformed by any suitable technique including, but not limited to, joining together portions of the article using refastenable and/or nonrefastenable bonds (e.g., seam, weld, adhesive, cohesive bond, fastener, etc.). A pant may be preformed anywhere along the circumference of the article (e.g., side fastened, front waist fastened).

“Polymer” as used herein refers to but is not limited to, homopolymers, copolymers, such as for example, block, graft, random and alternating copolymers, terpolymers, etc. and blends and modifications thereof. Unless otherwise specifically limited, the term “polymer” includes all possible spatial configurations of the molecule and include, but are not limited to isotactic, syndiotactic and random symmetries.

“Rear” as used herein refers to the portion of an absorbent article or part thereof that is intended to be positioned proximate the back of the wearer.

“Release structure”, “release region”, “release surface” or “release material” and the like as used herein are used interchangeably and refer to a structure in fluid communication with the absorbent core having a larger relative liquid absorption capacity and/or rate allowing it to quickly take up, temporarily hold and releasing liquids.

“Resin” as used herein refers to a solid or semisolid polymeric material.

“Thermobonding” as used herein refers to a method of bonding fibers by the use of heat and/or high-pressure.

“Thermoplastic” as used herein refers to polymeric materials that have a melting temperature and can flow or be formed into desired shapes on the application of heat at or below the melting point.

“Ultrasonic” as used herein refers to the use of high frequency sound to generate localized heat through vibration thereby causing thermoplastic fibers to bond to one another.

“Water-absorbing”, “liquid-absorbing”, “absorbent”, “absorbing” and the like as used herein are used interchangeably and refer to compounds, materials, products that absorb at least water, but typically also other aqueous fluids and typically other parts of bodily exudates such as at least urine or blood.

“Wearer facing layer” as used herein refers to elements of the chassis that form the inner surface of the absorbent article, such as the topsheet, the leg cuffs, and the side panels, etc., when such elements are present.

“Weaving” as used herein refers to the process of interlacing two or more sets of yarns at right angles to form a fabric; a web of fibers produced by weaving is herein referred to as a “woven”.

“Web material” as used herein refers to an essentially endless material in one direction, i.e. the longitudinal extension or the length, or the x-direction in Cartesian coordinates relative to the web material. Included in this term is an essentially unlimited sequence of pieces cut or otherwise separated from an essentially endless material. Often, though not necessarily, the web materials will have a thickness dimension (i.e. the z-direction) which is significantly smaller than the longitudinal extension (i.e. in x-direction). Typically, the width of web materials (they-direction) will be significantly larger than the thickness, but less than the length. Often, though not necessarily, the thickness and the width of such materials is essentially constant along the length of the web. Without intending any limitation, such web materials may be cellulosic fiber materials, tissues, woven or nonwoven materials and the like. Typically, though not necessarily, web materials are supplied in roll form, or on spools, or in a folded state in boxes. The individual deliveries may then be spliced together to form the essentially endless structure. A web material may be composed of several web materials, such as multilayer non-woven, coated tissues, nonwoven/film laminates. Web materials may comprise other materials, such as added binding material, particles, hydrophilizing agents and the like.

“Wet burst strength” is a measure of a layer's ability to absorb energy, when wet and subjected to deformation normal to the plane of the web.

“Wet strength” as used herein refers to the strength of a joint determined immediately after removal from a liquid in which it has been immersed under specified conditions of time, temperature and pressure. The term is commonly used in the art to designate strength after immersion in water.

“Wetlaying” as used herein refers to the forming a web from an aqueous dispersion of fibers by applying modified paper making techniques; a web of fibers produced by wetlaying is herein referred to as a “wetlaid”.

“Wood pulp” as used herein refers to cellulosic fibers used to make viscose rayon, paper and the absorbent cores of products such as feminine hygiene garments, baby diapers and pants and adult incontinence garments.

“X-y dimension” as used herein refers to the plane orthogonal to the thickness of the article, structure or element. The x-and y-dimensions correspond generally to the width and length, respectively, of the article, structure or element.

“Z-dimension” as used herein refers to the dimension orthogonal to the length and width of the article, structure or element. The z-dimension corresponds generally to the thickness of the article, structure or element.

1. A package (1000) comprising a bag (100) and a stack of absorbent articles (200) having a substantially rectangular shape, wherein the stack of absorbent articles is arranged in the bag, wherein the bag (100) is made of a paper material, wherein the bag (100) has a closed bottom (110), a closed top (120), at least one outer attached portion (180), and a peripheral wall (130) between said bottom and said top, wherein the bag is provided at least on an inner surface of the bag near the top, with a first sealable coating (140), wherein the closed top (120) is realized by a sealed portion (145) of said first sealable coating (140), wherein the bag is provided at least on an outer surface of the bag near the top with a second sealable coating (190) wherein the at least one outer attached portion (180) is realized by a sealed portion (195) of the second sealable coating (190).

2. The package of claim 1, wherein the peripheral wall (130) comprises a front wall (133), a rear wall (134) opposite said front wall, a first side wall (131), and a second side wall (132) opposite said first side wall, and wherein the second sealable coating (190) is provided on the first side wall (131) or the second side wall (132) to realize the at least one outer attached portion (180) on the first side wall (131) or the second side wall (132), respectively.

3. The package of claim 1, wherein the peripheral wall (130) comprises a front wall (133), a rear wall (134) opposite said front wall, a first side wall (131), and a second side wall (132) opposite said first side wall, wherein the at least one outer attached portion (180) comprises a first outer attached portion (180a) and a second outer attached portion (180b) and wherein the second sealable coating (190) is provided on the first side wall (131) and the second side wall (132) to realize the first outer attached portion (180a) on the first side wall (131) and the second attached portion (180b) on the second side wall (132).

4. The package of claim 2 or 3, wherein the first side wall (131) or/and the second side wall (132) is folded inwardly at the location of the second sealable coating (190) such that two first folded portions (121) are formed where the top (120) joins the first side wall (131) or/and such that two second folded portions (122) are formed where the top (120) joins the second side wall (132), and wherein the at least one outer attached portion (180) is realized by a sealed portion (195) of the second sealable coating (190) attaching the two first folded portions together or/and attaching the two second folded portions (122) together, respectively.

5. The package of any one of the preceding claims, wherein the paper material has a basis weight between 40 and 100 g/m², preferably between 50 and 90 g/m², more preferably between 60 and 80 g/m², measured according to the ISO 536 standard.

6. The package of any one of the preceding claims, wherein the first sealable coating (140) extends at least over a closed peripheral inner portion of the inner surface of the bag (100).

7. The package of any one of the preceding claims, wherein the second sealable coating (190) is present on the outer surface of the bag at least at the level of the first sealable coating of the wing portion or folded portion, wherein the first sealable coating of the wing portion or folded portion is present on the inner surface of the bag.

8. The package of any one of the previous claims, wherein the stack of absorbent articles is arranged such that each absorbent article (200) extends in an upright direction between the bottom (110) and the top (120).

9. The package of any one of the previous claims, wherein the absorbent article is a folded absorbent article and wherein a crotch portion (201) of the absorbent article is located at the top (120) of the bag, and wherein a rear and front end (202, 203) of the absorbent article are located at the bottom (110) of the bag.

10. The package of any one of the previous claims, wherein the absorbent articles are folded absorbent articles having a width (wa) between 7 cm and 20 cm, and a height (ha) between 15 cm and 40 cm.

11. The package of any one of the previous claims, wherein the bottom has a width (w) between 9 cm and 20 cm, preferably between 10 cm and 15 cm, and a length (1) between 15 cm and 50 cm, preferably between 20 cm and 40 cm.

12. The package of claim 4 and any one of the previous claims, wherein the length (ls1) of the attached first folded portions and the length (ls2) of the attached second folded portions is between 1 cm and 8 cm, preferably between 2 cm and 7 cm, more preferably between 3 cm and 6 cm.

13. The package of claim 4 and any one of the previous claims wherein the length (ls) of the sealed portion between the attached first folded portions and the attached second folded portions is between 8 cm and 40 cm, preferably between 10 cm and 30 cm, more preferably between 12 cm and 20 cm.

14. The package of any one of the previous claims, wherein the first sealable coating (140) is made of any one of the following materials or a combination thereof: a polymer material such as a polyethylene material, e.g. as a low density polyethylene (LDPE), a bio coating, a printed lacquer, 1,4 succinic, fumaric and malic acids, 2,5 furan dicarboxylic acid, 3hydroxy propionic acid, aspartic acid, glucaric acid, glutamic acid, itaconic acid, levulinic acid, 3-hydroxybutyrolactone, glycerol, sorbitol, xylitol/arabinito, tricarboxylic acid.

15. The package of any one of the previous claims, wherein the first sealable coating (140) has a basis weight between 5 g/m² and 25 g/m², preferably between 7 g/m² and 20 g/m², more preferably between 8 g/m² and 16 g/m².

16. The package of any one of the previous claims, wherein the second sealable coating (190) is made of any one of the following materials or a combination thereof: a polymer material such as a polyethylene material, e.g. as a low density polyethylene (LDPE), a bio coating, a printed lacquer, 1,4 succinic, fumaric and malic acids, 2,5 furan dicarboxylic acid, 3hydroxy propionic acid, aspartic acid, glucaric acid, glutamic acid, itaconic acid, levulinic acid, 3-hydroxybutyrolactone, glycerol, sorbitol, xylitol/arabinito, tricarboxylic acid.

17. The package of any one of the previous claims, wherein the second sealable coating (190) has a basis weight between 5 g/m² and 25 g/m², preferably between 7 g/m² and 20 g/m², more preferably between 8 g/m² and 16 g/m².

18. The package of any one of the previous claims, wherein the paper material has a basis weight measured according to the ISO 536 standard between 65 g/m² and 85 g/m².

19. The package of any one of the previous claims, wherein the bottom is formed by folding and gluing a portion of the paper material, such that is has a substantially rectangular shape.

20. The package of any one of the previous claims, wherein the bottom is provided with a handle (155) made of paper material; and/or
wherein the package further comprises a paper sheet (150) with two cuts (153), said paper sheet being glued to the bottom of the bag in an area (151) around the two cuts, such that an area (152) of the paper sheet between the two cuts functions as the handle (155).

21. The package of any one of the previous claims, wherein the paper material is an unbleached paper material.

22. The package of any one of the previous claims, wherein the paper material has any one or more following properties, in test conditions as specified in ISO 554-1976 (23+/−1° C./50±/−2%):
a. a tensile strength measured according to the ISO 1924-3 standard in a machine direction between 4 and 10 kN/m, preferably between 5 and 7 kN/m, and in a cross direction between 2 and 6 kN/m, preferably between 4 and 5 kN/m;
b. a tear index measured according to the ISO 1974 standard in a machine direction between 8 and 16 mN.m2 /g, and in a cross direction between 9 and 17 mN.m2 /g;
c. a tear measured according to the ISO 1974 standard in a machine direction between 400 and 1300 mN, and in a cross direction between 450 and 1450 mN.

23. A method for manufacturing a package for a stack of absorbent articles, comprising the steps of:
a. arranging the stack of absorbent articles (200) in a bag (100′) made of a paper material; said bag (100′) having a closed bottom (110), an open top (120′), and a peripheral wall (130) between said bottom and said top; wherein the bag is provided at least on an inner surface of the bag near the top, with a first sealable coating (140), and wherein the bag is provided at least on an outer surface of the bag near the top, with a second sealable coating (190);
b. sealing a portion (145) of said first sealable coating (140) to obtain a closed top end (120); and
c. sealing a portion (195) of said second sealable coating (190) to obtain at least one outer attached portion (180).

24. The method of the previous claim, wherein the peripheral wall (130) comprises a front wall (133), a rear wall (134) opposite said front wall, a first side wall (131), and a second side wall (132) opposite said first side wall, and wherein, before steps b. and c., the first and the second side wall are folded inwardly at the top (120), such that two first folded portions (121) are formed where the top (120) joins the first side wall (131) or/and such that two second folded portions (122) are formed where the top (120) joins the second side wall (132), and wherein the at least one outer attached portion (180) is realized by a sealed portion (195) of the second sealable coating (190) attaching the two first folded portions together or/and attaching the two second folded portions (122) together, respectively.

25. The method of any one of the preceding claims 23-24, wherein, before the steps b. and c., the open top end (120′) of the bag is provided with a manipulation part (160, 960) containing a plurality of holes (161), and wherein, prior to the step of arranging of the stack of absorbent articles in the bag, the bag is arranged on a stack of bags, with a plurality of pins (301) extending through the plurality of holes (161), and wherein the step of arranging comprises opening the bag (100′) whilst the bag is held by the plurality of pins (301) and pushing the stack of absorbent articles (200) in the bag (100′).

26. The method of claim 25, wherein the plurality of pins comprises a plurality of retarding elements (710, 810), and wherein during opening of the bag (100′) the plurality of holes (161) of the manipulation part (160, 960) are guided over the plurality of retarding elements.

27. The method of the preceding claim, wherein each retarding element of the plurality of retarding elements comprises a first portion (710a, 810a) having a first cross-section and a second portion (710b, 810b) having a second cross-section, wherein the first cross-section is smaller than the second cross-section/circumference.

28. The method of the preceding claim, wherein during opening of the bag (100′) each hole of the plurality of holes is guided over the first portion of the respective retarding element before being guided over the second portion of the respective retarding element.

29. The method of any one of the preceding claims 27-28, wherein a cross-section of the retarding element gradually increases from the first cross-section to the second cross-section.

30. The method of any one of the preceding claims 25-29, wherein the manipulation part (160) containing the plurality of holes is a part made of a paper material, e.g. a paper material having a basis weight between 40 and 100 g/m², preferably between 50 and 90 g/m², more preferably between 60 and 80 g/m², measured according to the ISO 536 standard, or of a polymer material, e.g. polyethylene material such as a low density polyethylene material.

31. The method of any one of the preceding claims 25-30, wherein the manipulation part (160) is a separate part glued to the bag (100′).

32. The method of any one of the preceding claims 25-30 wherein the manipulation part (160) is an integral part of the bag (100′).

33. The method of any one of the preceding claims 25-32, wherein the manipulation part (160) is substantially rectangular and has a length (lm) between 20 cm and 35 cm and a width (wm) between 4 cm and 15 cm.

34. The method of any one of the preceding claims 23-33, wherein the method further comprises, after or during the step of sealing, the step of cutting of an end part (170) of the top (120′) of the bag.

35. The method of any one of the preceding claims 25-34, wherein the end part (170) comprises the manipulation part (160) containing the plurality of holes.
